(19) United States
(12) Patent Application Publication
(10) Pub. No.: US 2015/0320300 A1
(43) Pub. Date: Nov. 12, 2015

(54) Systems and Methods of Distributing Illumination for Multiple Viewing Element and Multiple Illuminator Endoscopes

(71) Applicant: EndoChoice, Inc., Alpharetta, GA (US)

(72) Inventors: Yuri Gershov, Haifa (IL); Leonid Krivopisk, Nesher (IL)

(21) Appl. No.: 14/705,355

(22) Filed: May 6, 2015

Related U.S. Application Data
(60) Provisional application No. 61/989,895, filed on May 7, 2014.

(51) Int. Cl. A61B 1/045; A61B 1/06; A61B 1/00; A61B 1/05

(52) U.S. Cl. CPC A61B 1/045; A61B 1/05; A61B 1/0676; A61B 1/0623; A61B 1/00039; A61B 1/00006; A61B 1/00096; A61B 1/00009; A61B 1/0684

(57) ABSTRACT

A lens actuation system for an endoscope comprises at least one viewing element configured to capture images, a plurality of illuminators configured to illuminate a plurality of fields of view (FOVs) associated with the at least one viewing element, a controller, and actuatable lenses positioned in front of at least one of the plurality of illuminators and/or in front of the at least one viewing element. The actuatable lenses are configured to change dynamically a) an illumination direction of the at least one of the plurality of illuminators, and/or b) a direction of incoming light beams to the at least one viewing element.

### CROSS-REFERENCE TO RELATED APPLICATIONS

The present application relies on, for priority, U.S. Provisional Patent Application No. 61/989,895, entitled “Multi-Illuminator Endoscopic Lens Actuation Systems” and filed on May 7, 2014, which is herein incorporated by reference in its entirety.

The present application relates to U.S. patent application Ser. No. 14/603,137, entitled “Image Capture and Video Processing Systems and Methods for Multiple Viewing Element Endoscopes”, filed on Jan. 22, 2015, which relies on U.S. Provisional Patent Application No. 61/930,101, entitled “Daisy Chain Multi-Sensor Endoscopic System” and filed on Jan. 22, 2014 and U.S. Provisional Patent Application No. 61/948,012, entitled “Parallel Illuminating Systems” and filed on Mar. 4, 2014.

The present application also relates to U.S. patent application Ser. No. 13/655,120, entitled “Multi-Viewing Element Endoscope”, and filed on Oct. 18, 2012.

In addition, the present application also relates to U.S. patent application Ser. No. 13/882,004, entitled “Optical System for Multi-Sensor Endoscopes”, filed on Apr. 26, 2013, which is a 371 National Stage Entry of PCT Application Number PCT/IL11/000832, of the same title, and filed on Oct. 27, 2011, which, in turn, relies upon U.S. Provisional Patent Application No. 61/407,495, filed on Oct. 28, 2010.

The present application also relates to U.S. patent application Ser. No. 13/992,014, entitled “Flexible Electronic Circuit Board for a Multi-Camera Endoscope”, filed on Jun. 6, 2013, which is a 371 National Stage Entry of PCT Application Number PCT/IL11/050049, of the same title, and filed on Dec. 8, 2011, which, in turn, relies upon U.S. Provisional Patent Application No. 61/421,238, filed on Dec. 9, 2010.

All of the above-mentioned applications are herein incorporated by reference in their entirety.

### FIELD

The invention relates generally to endoscopy systems and, in particular, to image capture and video processing systems and methods in multiple-illuminator endoscope systems.

### BACKGROUND

Free space is an extremely valuable resource within a multiple camera endoscope tip section. Such tip sections typically include a plurality of cameras, a plurality of optical systems, a plurality of illuminators, a flexible electronic circuit board configured to support and encapsulate the components and a working channel configured for the injection of fluids and for the insertion of miniscule surgery tools.

An optical system for a tip section of a multiple sensor endoscope comprising a front-pointing camera sensor, a front objective lens system, a side-pointing camera-sensor, and a side objective lens system is disclosed in U.S. patent application Ser. No. 13/882,004, entitled “Optical Systems for Multi-Sensor Endoscopes” and filed on May 23, 2013, which is herein incorporated by reference in its entirety.

A flexible electronic circuit board for a multiple camera endoscope tip section is disclosed in Patent Cooperation Treaty Application Number PCT/IL2011/050049, entitled “Flexible Electronic Circuit Board for a Multi-Camera Endoscope” and filed on Dec. 8, 2011, which is herein incorporated by reference in its entirety. The circuit board comprises: a front camera surface configured to carry a forward looking camera; a first side camera surface configured to carry a first side looking camera; a second side camera surface configured to carry a second side looking camera; one or more front illuminator surfaces configured to carry one or more front illuminators; and, one or more side illuminators surfaces configured to carry one or more side illuminators.

The flexible circuit board is connected to the central control unit via a multi-wire cable. The multi-wire cable is welded on the board in a designated location, freeing additional space within the tip section assembly and adding flexibility to the cable access.

A multiple sensor or multiple viewing elements endoscope tip section comprising a front-pointing camera and two or more side-pointing cameras positioned at or in proximity to a distal end of the tip section and a working channel configured for insertion of a surgical tool is disclosed in U.S. patent application Ser. No. 13/655,120, entitled “Multi-Camera Endoscope” and filed on Oct. 18, 2012, which is herein incorporated by reference in its entirety, and assigned to the Applicant of the present specification. As described in the '120 application, the field of view (FOV) of each camera sensor in a multiple sensor endoscope is illuminated by two or more illuminators that are light emitting diodes (LEDs). Thus, multiple sensor endoscopes' tips that include a right pointing camera or viewing element, a front pointing camera or viewing element and a left pointing camera or viewing element may include a minimum of 9 or more LEDs. Since the FOVs' depth in different orientations, for example in a patient's colon, can vary significantly during a colonoscopy procedure, illuminating all LEDs with a fixed illumination intensity is sub-optimal, may be too weak in some orientations for example and may drive the camera sensor arrays beyond their dazzle limits due to light reflection from a nearby wall in other orientations.

One approach for controlling the illumination of a multiple illuminator endoscope system may be provided by dynamically controlling the emitted light intensities. However, since multiple illuminator endoscope systems may include 10 or more illuminators, controlling the light intensity of each illuminator independent of the other illuminators dynamically may be a difficult task. Another approach for controlling the illumination of multiple illuminator endoscope systems is provided by dynamically actuating electro and/or electro-mechanical actuatable lenses.

It would also be highly advantageous to provide lens actuation systems used to dynamically redirect the illumination of multiple illuminator endoscopes.

### SUMMARY

The present specification discloses a lens actuation system for an endoscope, the system comprising: at least one viewing element configured to capture images; a plurality of illuminators configured to illuminate a plurality of fields of view (FOVs) associated with the at least one viewing element; a controller; and at least one actuatable lens positioned in front of one of the plurality of illuminators, wherein the at least one actuatable lens is configured to dynamically change an illumination direction of the one of the plurality of illuminators based upon a signal from said controller.

Optionally, the lens actuation system comprises a front viewing element with at least two front illuminators and a side viewing element with at least two side illuminators, wherein an actuatable lens is positioned in front of each of the two front illuminators and the two side illuminators to dynamically change an illumination direction of one or both illuminators of the front and side illuminators based upon a signal from the controller.

Optionally, the lens actuation system comprises a front viewing element with at least two front illuminators and a side viewing element with at least two side illuminators, wherein an actuatable lens is positioned in front of the front and side viewing elements to dynamically change direction of incoming light beams to the front and side viewing elements based upon a signal from said controller.

The present specification also discloses a lens actuation system for an endoscope, the system comprising: at least one viewing element configured to capture images; a plurality of illuminators configured to illuminate a plurality of fields of view (FOVs) associated with the at least one viewing element; a controller; and an actuatable lens positioned in front of the at least one viewing element, wherein the actuatable lens is configured to dynamically change direction of incoming light beams, from the plurality of FOVs to the at least one viewing element, based upon a signal from the controller.

The present specification also discloses a lens actuation system for an endoscope, the system comprising: at least one viewing element configured to capture images; a plurality of illuminators configured to illuminate a plurality of fields of view (FOVs) associated with the at least one viewing element; a controller; and actuatable lenses positioned in front of the at least one viewing element and in front of at least one of the plurality of illuminators, wherein, based upon a signal from the controller, the actuatable lenses are configured to dynamically change one or both of a) an illumination direction of said at least one of said plurality of illuminators, and b) a direction of incoming light beams to said at least one viewing element,

The plurality of FOVs may partially overlap.

An actuatable lens (or lenses) may be positioned in front of each of the plurality of illuminators, or an actuatable lens (or lenses) may be positioned in front of more than one of the plurality of illuminators.

Optionally, the actuatable lens (or lenses) is a stiff lens positioned proximate to a plurality of electro actuators configured to move the stiff lens.

Optionally, the actuatable lens (or lenses) is a flexible lens positioned proximate to a plurality of actuators configured to deform the flexible lens. Optionally, the flexible lens is a silicon lens and the deformation of the flexible lens is selected from a group consisting of: contracting, expanding, pulling, pushing and combinations thereof.

The actuatable, lens (or lenses) may be coated with an electro responsive material that changes its local light refraction index in response to an applied electric field, Optionally, the electro responsive coating material is selected from a group consisting of: a liquid crystal, an electro responsive polymer, inorganic crystals, metamaterials or combinations thereof. Optionally, the electric field is applied by a plurality of electrodes.

Optionally, the actuatable lens (or lenses) dynamically changes an illumination direction by rotating or translating or a combination thereof.

Optionally, where light intensity exceeds a predetermined threshold at a certain FOV, due to at least a partial overlap of illumination from more than one of the plurality of illuminators, the at least one actuatable lens is configured to dynamically redirect illumination from at least one of the plurality of illuminators in order to reduce light intensity at the certain FOV.

Optionally, where light intensity is below a predetermined threshold at a certain FOV, the at least one actuatable lens is configured to dynamically redirect illumination from at least one of the plurality of illuminators in order to increase light intensity at the certain FOV.

The at least one actuatable lens may be actuated based on a detection of bright and/or dark areas in the plurality of FOVs.

Optionally, a user interface is configured to allow a user to manually actuate the at least one actuatable lens according to desired light intensity of images presented on a display.

Optionally, a user interface is configured to allow a user to actuate the at least one actuatable lens in order modify a blooming effect, saturation effect, underexposure effect, or overexposure effect.

The present specification also discloses a method of controlling illumination of an endoscope tip, the method comprising: providing, at the endoscope tip, at least one viewing element configured to capture images, a plurality of illuminators configured to illuminate a plurality of field of views (FOVs) associated with the at least one viewing element and at least one actuatable lens positioned in front of at least one of the plurality of illuminators; receiving, from the at least one viewing element, images of a plurality of FOVs illuminated by the plurality of illuminators; and dynamically redirecting illumination by actuating the at least one actuatable lens in order to reduce light intensity of a too bright captured image or increase light intensity of a too dark captured image.

The aforementioned and other embodiments of the present specification shall be described in greater depth in the drawings and detailed description provided below.

### BRIEF DESCRIPTION OF THE DRAWINGS

These and other features and advantages of the present specification will be further appreciated, as they become better understood by reference to the detailed description when considered in connection with the accompanying drawings:

FIG. 1 illustrates an exemplary tip section of an endoscope that includes a plurality of viewing elements and a parallel illuminating systems, according to certain embodiments of the present specification;

FIG. 2 shows a multiple illuminator and viewing elements endoscopy system, according to some embodiments;

FIG. 3A is a block diagram showing an LED, an actuatable lens and piezoelectric actuators, according to certain embodiments of the present specification;

FIG. 3B is a block diagram of a multiple illuminator endoscope system that includes at least one viewing element, at least one illuminator, optical assemblies and a control unit, according to certain embodiments of the present specification;

FIG. 4A illustrates an exemplary tip section of an endoscope, showing a bright central illumination FOV, according to certain embodiments of the present specification;

FIG. 4B illustrates the tip section of FIG. 4A, showing a redirection of the illumination FOV of at least one illuminator, via lens actuation, according to certain embodiments of the present specification;

FIG. 5 illustrates a stiff lens actuation system as used in a multiple illuminator endoscope, according to some embodiments of the present specification;

FIG. 6 illustrates a flexible lens actuation system as used in a multiple illuminator endoscope, according to some embodiments of the present specification;

FIG. 7 illustrates an electro-responsive coated lens actuation system, as used in a multiple illuminator endoscope, according to some embodiments of the present specification; and

FIG. 8 is a flow chart illustrating a plurality of exemplary steps of a method of controlling distribution of illumination or brightness for a multiple illuminator endoscopic tip of the present specification, in accordance with an embodiment.

### DETAILED DESCRIPTION

In the description and claims of the present specification, each of the words “comprise”, “include”, and “have”, and forms thereof, are not necessarily limited to members in a list with which the words may be associated.

The present specification is directed toward multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present specification is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

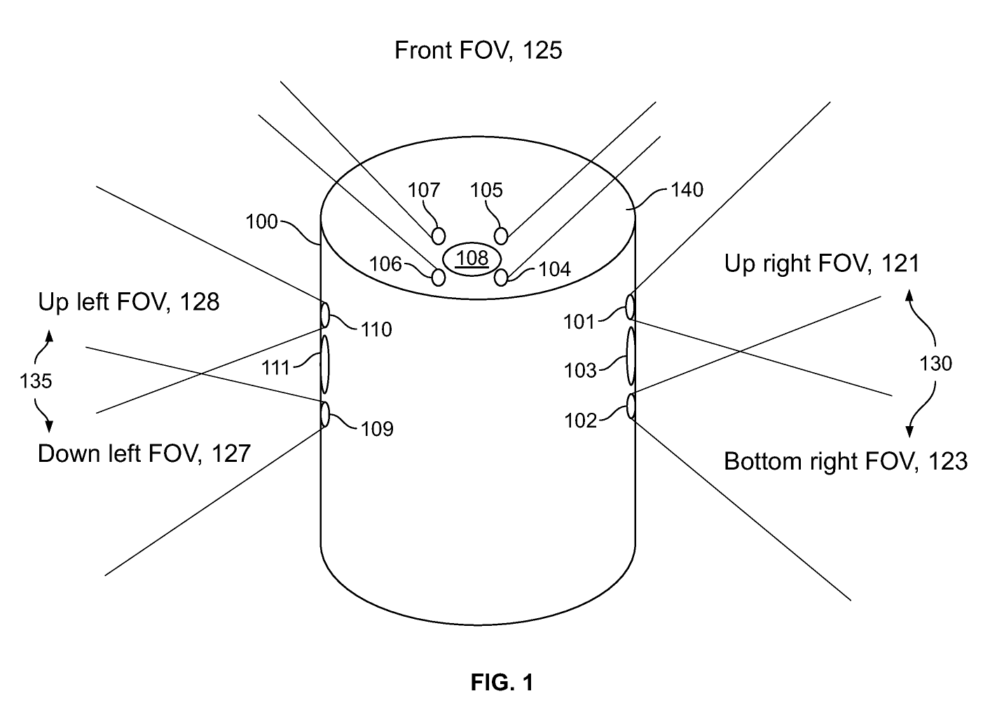

Reference is now made to FIG. 1, which illustrates an exemplary endoscope tip section 100 comprising a plurality of viewing elements, also referred to as cameras, sensors or camera sensors, and a parallel illuminating system comprising a plurality of illuminators associated with the plurality of viewing elements, according to certain embodiments. The parallel illuminating system comprises a side pointing viewing element 103 and two associated side pointing illuminators 101 and 102 respectively illuminating an upper right field of view (FOV) 121 and a lower right FOV 123 (the FOVs 121 and 123 may partially overlap in various embodiments) to together illuminate a right FOV 130; a front pointing viewing element 108 and four associated front pointing illuminators 104, 105, 106 and 107 (the FOVs of the four front pointing illuminators may partially overlap in various embodiments), which together illuminate a front FOV 125; and another side pointing viewing element 111 and two associated side pointing illuminators 109 and 110 (the FOVs 109, 110 may partially overlap in various embodiments), which respectively illuminate a lower left FOV portion 127 and an upper left FOV portion 128, together illuminating a left FOV 135.

In accordance with various embodiments, viewing elements, cameras or sensors 103, 108 and 111 are Charge Coupled Device (CCD) or Complementary Metal Oxide Semiconductor (CMOS) image sensor arrays. Also, front illuminators 104, 105, 106, 107 and side illuminators 101, 102, 109, 111 are, in an embodiment, discrete illuminators and include a light-emitting diode (LED), which may be a white light LED, an infrared light LED, a near infrared light LED, an ultraviolet light LED or any other LED. The term “discrete”, concerning discrete illuminator, refers to an illumination source, which generates light locally and internally, in contrast to a non-discrete illuminator, which may be, for example, a fiber optic merely transmitting light generated remotely.

It should be understood that the endoscope tip section 100 includes a working channel, having an opening positioned on the front face 140 that is configured to inject fluids or gases and to insert surgical tools, a plurality of optical systems that may include front and side objective lens systems, a flexible electronic circuit board configured to carry the front and side viewing elements along with the associated illuminators, the wiring connections between these components and a cable connecting the parallel illuminating system of the endoscopic tip 100 to an endoscope handle, to an external control unit and to a display.

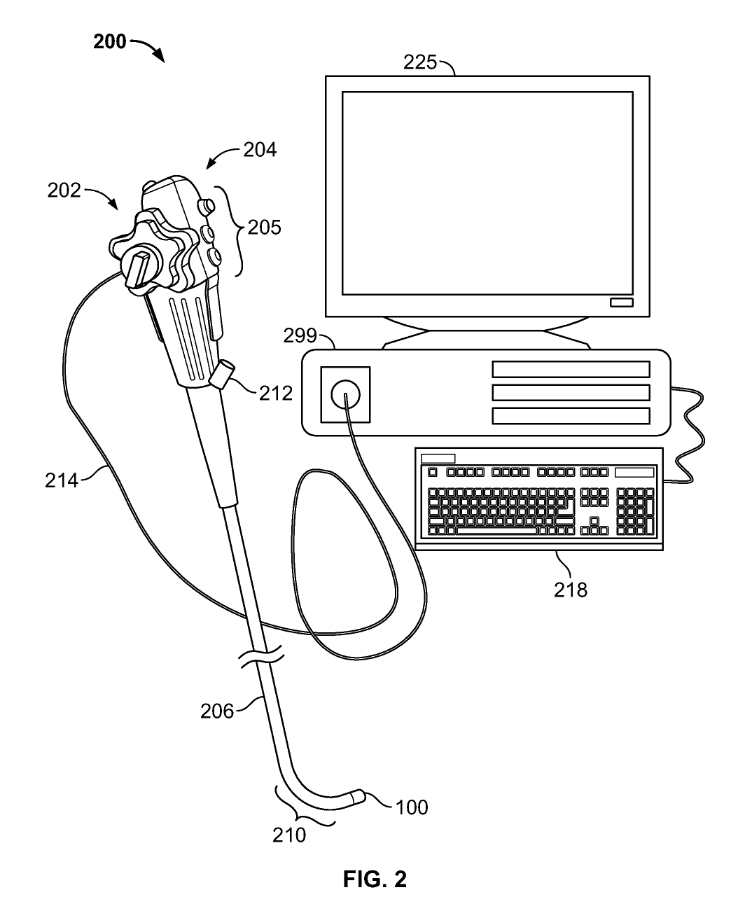

Reference is now made to FIG. 2, which shows a multiple viewing elements endoscopy system 200. System 200 includes a multiple viewing elements endoscope 202. The multiple viewing elements endoscope 202 includes a handle 204, from which an elongated shaft 206 emerges. Elongated shaft 206 terminates with a tip section 100 (as shown in FIG. 1) which is turnable by way of a bending section 210. The handle 204 is used for maneuvering elongated shaft 206 within a body cavity. The handle 204 includes one or more buttons, knobs and/or switches 205 which control bending section 210 as well as functions such as fluid injection and suction. Handle 204 further includes at least one working channel opening 212 through which surgical tools may be inserted. In some embodiments, the handle 204 also includes one or more side service channel openings for one or more side service channels provided in the tip section 100.

A utility cable 214, also referred to as an umbilical tube, connects between the handle 204 and a Main Control Unit 299. Utility cable 214 includes therein one or more fluid channels and one or more electrical channels. The electrical channel(s) include at least one data cable for receiving video signals from the front and side-pointing viewing elements, as well as at least one power cable for providing electrical power to the viewing elements and to the discrete illuminators (that is, to the parallel illuminating system).

The main control unit 299 contains the controls required for displaying the images and/or videos of internal organs captured by the endoscope 202. The main control unit 299 governs power transmission to the endoscope's 202 tip section 100, such as for the tip section's viewing elements and illuminators. The main control unit 299 further controls one or more fluid, liquid and/or suction pump(s) which supply corresponding functionalities to the endoscope 202. One or more input devices 218, such as a keyboard, a touch screen and the like is connected to the main control unit 299 for the purpose of human interaction with the main control unit 299. In the embodiment shown in FIG. 2, the main control unit 299 comprises a screen/display 225 for displaying operation information concerning an endoscopy procedure when the endoscope 202 is in use. The screen 225 is configured to display images and/or video streams received from the viewing elements of the multiple viewing element endoscope 202. The screen 225 may further be operative to display a user interface for allowing a human operator to set various features of the endoscopy system.

It should be appreciated that the parallel illuminating system, illustrated in FIG. 1, is a non-limiting example. In alternate embodiments, the parallel illuminating system may have varying number of illuminators. Also, such varying number of illuminators may be associated with a front viewing element and only one side viewing element in various embodiments. According to still further embodiments of the present specification, similar parallel illuminating systems may be used in automotive industry, large display screens, in office and home illuminating systems and the like.

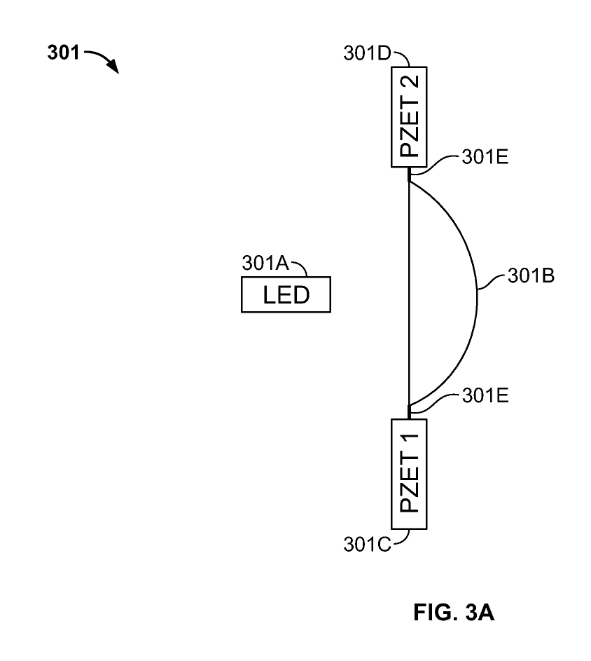

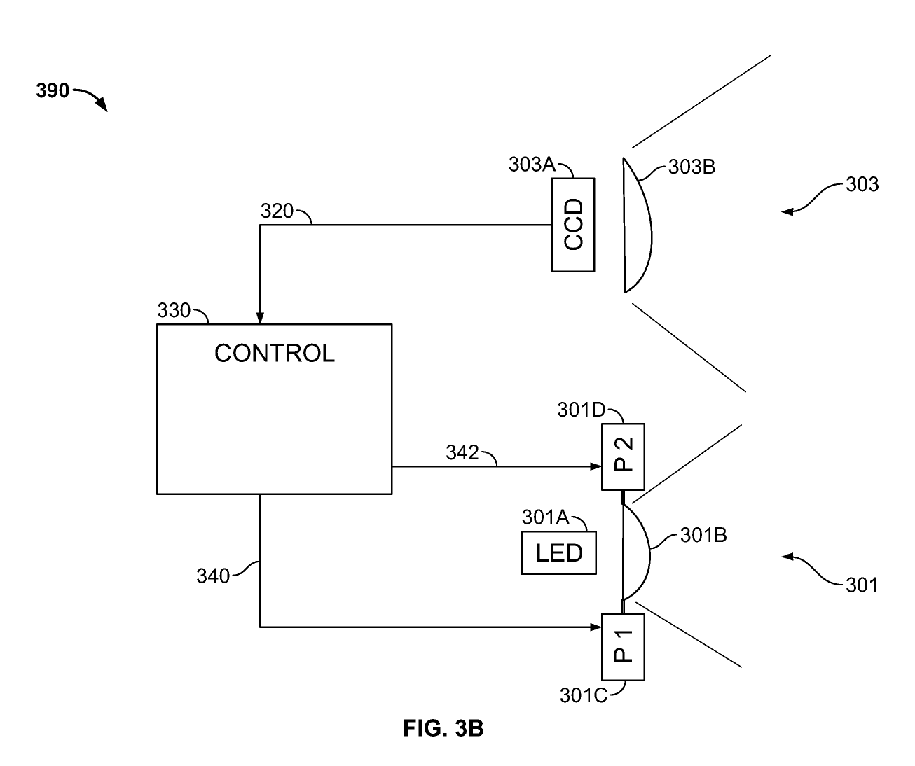

Reference is now made to FIG. 3A (along with FIG. 1), which illustrates an illuminator, such as an LED, an actuatable lens and piezoelectric actuators, according to certain embodiments. Illuminator 301, comprises an LED 301A, an associated actuatable lens 301B, piezoelectric actuators 301C and 301D and brackets 301E that, in various embodiments, are flexible brackets or supports of silicon or rubber, for example. Each illuminator, of the parallel illuminating system shown in FIG. 1 (101, 102, 104-107, 109 and 110), includes the components shown in FIG. 3A that are actuated by a central or main control unit (such as the main control unit 299 shown in FIG. 2).

Reference is now made to FIG. 3B, which illustrates a block diagram of a multiple illuminator endoscope that includes at least one viewing element or camera, at least one illuminator, optical assemblies and control unit, according to certain embodiments. Multiple illuminator endoscope system 390 comprises at least one viewing element or camera sensor 303A that includes actuatable or fixed lens 303B. The camera sensor 303A is configured to capture images received through lens 303B and to transmit, via line 320, the sampled images converted to digital pixel data to control unit 230. The figure also illustrates, as an example, the illuminator 301 along with its components described with reference to FIG. 3A. The control unit 330 is a central control unit (similar to the main control unit 299 of FIG. 2) configured to control a plurality of viewing elements or camera sensors, such as the viewing element 303A, and a plurality of illuminators along with the associated piezoelectric actuators, such as 301C, by control line 340, and 301D by control line 342. In one embodiment, the control unit 330 is configured to control the LED 301A and lens 303B where the camera lens 303B is an actuatable lens.

Persons of ordinary skill in the art should note that depending upon the type of actuatable lens the actuating devices or systems may vary. Thus, in various embodiments, depending upon whether the actuatable lens is stiff, flexible or coated (with an electro responsive coating) the actuating devices could be piezoelectric actuators, electrodes, other miniature electromechanical actuators, such as but not limited to, linear step motors or step motors with miniature gears or electromagnets or micro electro mechanical systems (MEMS), which include electromechanical mechanisms controlled by electrostatic field.

It should be appreciated that the viewing elements, that are typically CCD or CMOS image sensors, have phenomena such as saturation and blooming that affect both their quantitative and qualitative imaging characteristics. For example, if each individual pixel can be thought of as a well of electrons, then saturation refers to the condition where the well becomes filled. The amount of charge that can be accumulated in a single pixel is determined largely by its area. However, due to the nature of the potential well, which holds charge within a pixel, there is less probability of trapping an electron within a well that is approaching saturation. Therefore, as a well approaches its limit, the linear relationship between light intensity and signal degrades. As a result, the apparent responsivity of a saturated pixel drops. At saturation, pixels lose their ability to accommodate additional charge. This additional charge then spreads into neighboring pixels, causing them to either report erroneous values or also saturate. This spread of charge to adjacent pixels is known as blooming and appears as a white streak or blob in the image. The occurrence of blooming, in video images generated by a multiple viewing elements endoscope, results in loss of details in portions of the video image that is a serious cause of concern for a physician performing an endoscopic procedure.

Therefore, according to an aspect of the present specification, the outgoing illumination or FOVs of a plurality of illuminators of a multi illuminator endoscopic tip are maneuverable for dynamic redirection. According to another aspect of the present specification, a lens of at least one viewing element of the multi illuminator endoscope tip is also moveable or maneuverable to dynamically redirect incoming light beams from a plurality of FOVs to the viewing element.

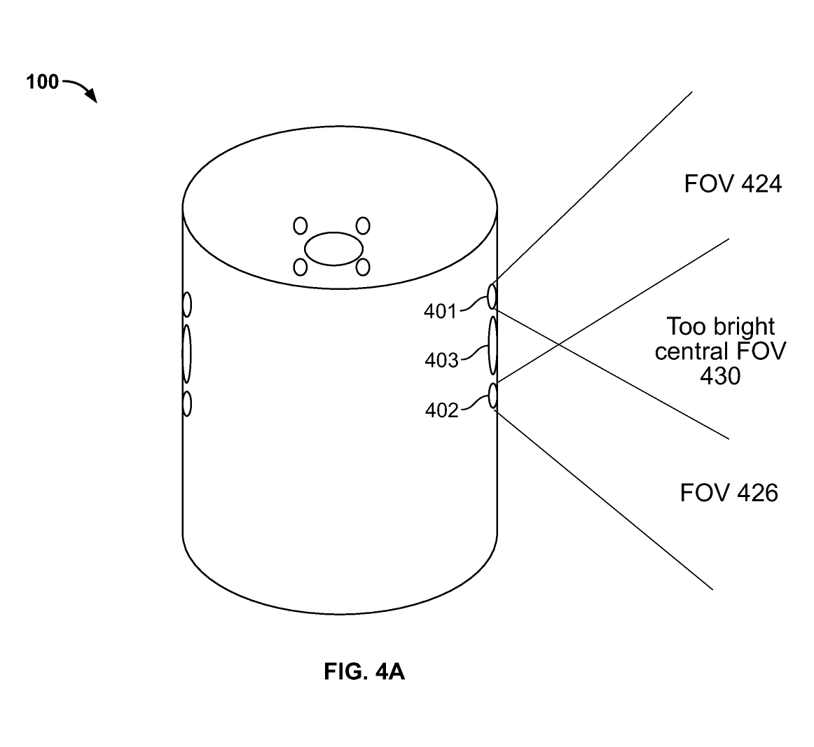

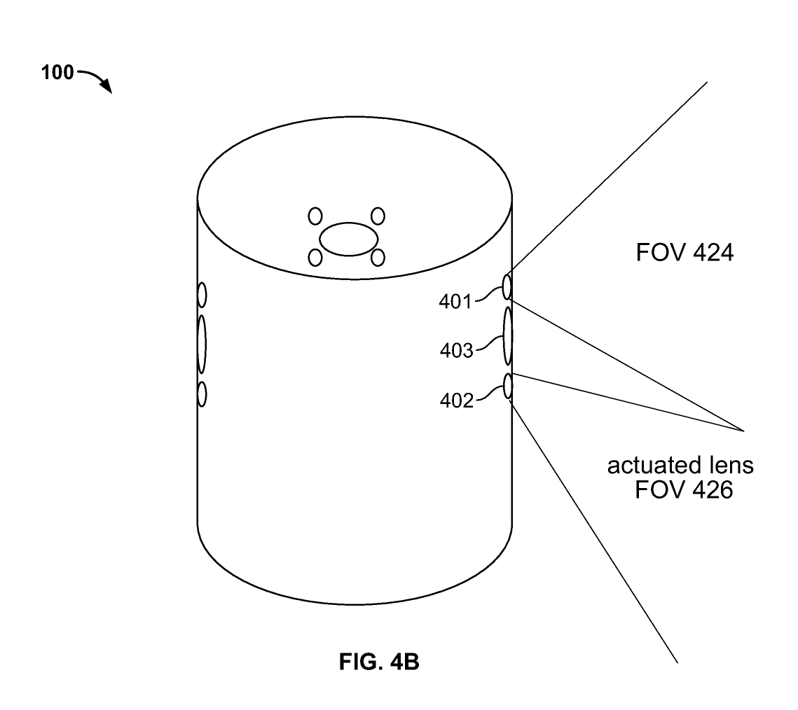

Reference is now made to FIG. 4A, which illustrates a bright central FOV 430, according to certain embodiments. The FOV 430 may encompass a wall of a patient's colon, for example, illuminated by the overlapping FOVs of the two illuminators 401 and 402. The combined illumination of illuminators 401 and 402, in the region of the overlapping FOV 430, may generate too bright a reflection (causing parts of an acquired image or video to be over exposed) captured by the viewing element 403. In other words, overlapping FOV 430 may over expose the viewing element or camera sensor 403 and other images that may appear in FOV 424 or 426 may be missed, partially or completely, due to the overly bright, over exposed, over lighted, saturated or bloomed reflection of overlapping FOV 430.

Reference is now made to FIG. 4B, which illustrates dynamically redirecting, by lens actuating or maneuvering, the illumination of at least one of the illuminators, according to certain embodiments. In accordance with an embodiment, the actuatable lens of the illuminator 402 is maneuvered or actuated such that the associated illumination or FOV 426 of the illuminator 402 is redirected to the right lower side of endoscope tip 100. This causes the FOVs 424 and 426 to not overlap thereby lowering or evenly distributing the illumination intensity of the two illuminators 401, 4102 across the combined FOVs 424, 426. Thus, the images captured by the viewing element 403 are evenly illuminated by the illuminators 401, 402.

According to an aspect of the present specification, a lens actuation system is used to dynamically redirect the illumination or FOV of at least one of the illuminators and/or of the lens of at least one viewing element, of a multi illuminator endoscopic tip. In various embodiments, the lens actuation system comprises an actuatable lens and piezoelectric actuators associated with an illuminator and/or with a lens of a viewing element. Referring back to FIG. 3A, for example, the lens actuation system of the LED 301A comprises the actuatable lens 301B and piezoelectric actuators 301C, 301D. Referring back to FIG. 3B, in some embodiments, the lens 303B of the viewing element 303 may also be actuatable in addition to the actuatable lens 301B of the illuminator 301.

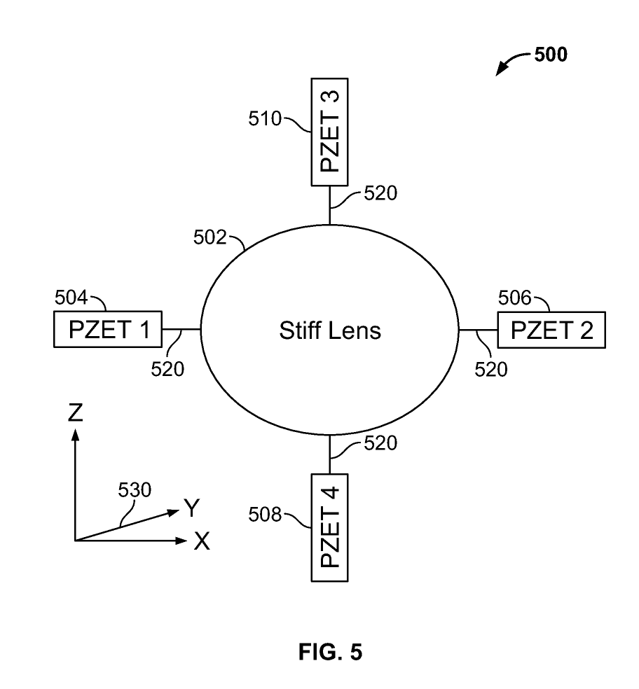

FIG. 5 illustrates a lens actuation system 500 in accordance with an embodiment. The lens actuation system 500 comprises a stiff lens 502 connected to a plurality of brackets, or holding or retaining elements, 520 that are further connected to devices capable of manipulating, deforming, or otherwise moving the lens, such as piezoelectric devices 504, 506, 508 and 510, other miniature electromechanical actuators, linear step motors, step motors with miniature gears, electromagnets, or MEMS. Each piezoelectric device 504, 506, 508 and 510 includes more than one piezoelectric device such that, upon electrical stimulation of one or more of the piezoelectric device 504, 506, 58, 510, each bracket 420 and therefore the connected stiff lens 502 may be moved or shifted in any one or more of the X, Y and Z directions 530 (the X, Y and Z directions being mutually orthogonal). Thus, in accordance with an embodiment, movements of the stiff lens 502 comprise rotations, translations or a combination thereof in any one or more of the three dimensions.

In some embodiments, the lens actuation system 500 is associated with a stiff lens of at least one illuminator, such as the lens 301B of the illuminator 301 of FIG. 3B, to dynamically redirect the outgoing illumination or FOV of the illuminator. In some embodiments, the lens actuation system 500 is associated with a stiff lens of at least one viewing element, such as the lens 303B of the viewing element 303 of FIG. 3B, to dynamically redirect incoming light beams from a plurality of FOVs to the at least one viewing element. In still further embodiments, the lens actuation system 500 is associated with a stiff lens of at least one illuminator as well as of at least one viewing element.

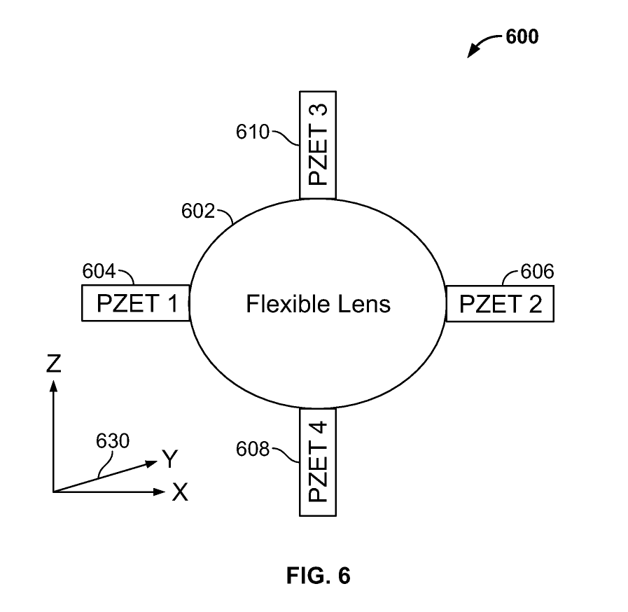

FIG. 6 illustrates a lens actuation system 600 in accordance with another embodiment. The lens actuation system 600 comprises a flexible lens 602 connected to a plurality of miniature electromechanical actuators such as piezoelectric devices 604, 606, 608 and 610. Each piezoelectric device 604, 606, 608 and 610 includes more than one piezoelectric device such that, upon electrical stimulation of one or more of the piezoelectric device 604, 606, 608, 610, the flexible lens 602 may be deformed in any one or more of the X, Y and Z directions 630 (the X, Y and Z directions being mutually orthogonal) by the actuating piezoelectric devices 604, 606, 608 and 610. In one embodiment, the flexible lens 602 is a silicon lens. Deformation of the flexible lens 602 comprises contracting, expanding, pulling and pushing of the flexible lens 602 in any one or more of the X, Y and Z directions 630.

In some embodiments, the lens actuation system 600 is associated with a flexible lens of at least one illuminator, such as the lens 301B of the illuminator 301 of FIG. 3B, to dynamically redirect the outgoing illumination or FOV of the illuminator. In some embodiments, the lens actuation system 600 is associated with a flexible lens of at least one viewing element, such as the lens 303B of the viewing element 303 of FIG. 3B, to dynamically redirect incoming light beams from a plurality of FOVs to the at least one viewing element. In still further embodiments, the lens actuation system 600 is associated with a flexible lens of at least one illuminator as well as with at least one viewing element.

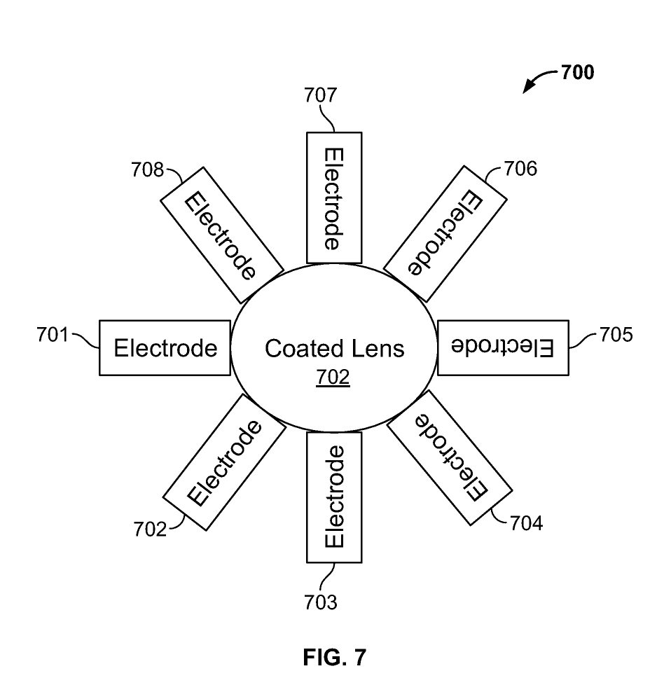

FIG. 7 illustrates a lens actuation system 700 in accordance with yet another embodiment. The lens actuation system 700 comprises an electro responsive coated lens 702 in electrical contact with a plurality of electrodes 701 to 708. Electrodes 701 to 708 are configured to apply an electric field onto the electro responsive coated lens 702 thereby varying, locally, the surface or volume refraction or attenuation index of the coated lens 702. The applied electric field, in voltage or voltage per meter, is used to dynamically redirect incoming or outgoing light beams through the coated lens 702 as a result of the modified refraction or attenuation index of the coated lens 702. The electro responsive coating material may be liquid crystal, an electro-responsive polymer, an inorganic crystal, a meta-material and combinations thereof. As used herein, meta-materials are artificial materials engineered to have properties that may not be found in nature.

In some embodiments, the lens actuation system 700 is associated with an electro responsive coated lens of at least one illuminator, such as the lens 301B of the illuminator 301 of FIG. 3B, to dynamically redirect the outgoing illumination or FOV of the illuminator. In some embodiments, the lens actuation system 600 is associated with an electro responsive coated lens of at least one viewing element, such as the lens 303B of the viewing element 303 of FIG. 3B, to dynamically redirect incoming light beams from a plurality of FOVs to the at least one viewing element. In still further embodiments, the lens actuation system 600 is associated with an electro responsive coated lens of at least one illuminator as well as with at least one viewing element.

Referring back to FIG. 2, the video controller or the controller circuit board of the main control unit 299 operatively connects with the endoscope 202 and the display units 225. The controller circuit board comprises elements for processing the video obtained from at least one viewing element, as well as other elements for system monitoring and control. Among the elements for processing the video are at least one DSP (to correspondingly process the image or video capture by the at least one viewing element) and an FPGA (Field Programmable Gate Array) that performs a plurality of logic tasks related to video stream or image processing—one of which includes detecting level of brightness such as under exposure and over exposure or blooming.

In accordance with an embodiment, the FPGA calculates the total average brightness of an image frame acquired by a viewing element. In another embodiment, the FPGA, around every luminance pixel sample arriving from the viewing element calculates a Gaussian (averaging process using Gaussian weighing) of its neighborhood pixels. Hence, the Gaussian is the local brightness around the luminance pixel sample. In alternate embodiment, however, the image processing, to determine the total average or Gaussian local brightness, is performed by a software program or by hardware processors such as an ASIC processor or a micro-controller and the like processing the data received from a viewing element. A local blooming control module, that calculates and uses a Gaussian local brightness to control blooming is described in U.S. Provisional Patent Application No. 62/093,871, entitled “System and Method for Processing Video Images Generated By A Multiple Viewing Elements Endoscope” and filed on Dec. 18, 2014, which is herein incorporated by reference in its entirety.

The total average brightness or the Gaussian local brightness, of an image or video frame, is compared with an upper threshold brightness level and a lower threshold brightness level. If the total average brightness or the Gaussian local brightness, of an image or video frame, is above the upper threshold brightness level it is indicative of an over exposed, over lighting, saturation or blooming regions within an image while if this is below the lower threshold brightness then it is indicative of under exposed, under lighted, too dark or dim regions. In one embodiment the upper and lower threshold brightness levels are pre-set by default (based on empirically determined optimal brightness preferences of a representative universal set of physicians). The range of acceptable brightness, as defined by the upper and lower thresholds, is further customizable by the physician depending upon his/her visual preference.

In one embodiment, areas or regions of an image or video frame having a Gaussian local brightness higher than the upper threshold brightness, intensity or luminance level are identified or segmented as being too bright, saturated or over exposed. Similarly, areas or regions of the image or video frame having, for example, Gaussian local brightness lower than the lower threshold brightness, intensity or luminance level are identified or segmented as being too dim or under exposed. Depending upon the viewing element that acquired the image or video frame, the main control unit automatically maneuvers the plurality of actuating devices associated with the plurality of illuminators that are used to illuminate the FOV of the viewing element. In one illustrative yet non-limiting example, as shown in FIGS. 4A and 4B, if the identified or segmented region is over exposed, such as in the overlapping FOV 426 of the two illuminators 401, 402 then the main control unit electrically stimulates the relevant actuating devices associated with the actuatable lens of at least one of the two illuminators so as to redirect the illumination FOV of one illuminator away with reference to the other illuminator till the Gaussian local brightness level of the identified over exposed region falls within the range defined by the upper and lower threshold brightness levels.

Similarly, if the identified or segmented region is under exposed, such as for example in the corners of the image then the actuatable lens of at least one illuminator (that illuminates the FOV of the viewing element responsible for capturing the image) is maneuvered to move the FOV of the at least one illuminator towards the under exposed region till the Gaussian local brightness level of the under exposed region falls within the range defined by the upper and lower threshold brightness levels. The actuatable lenses of one or more concerned illuminators are manipulated to ensure that the brightness or intensity levels of none of the regions or segments of the image or video frame fall outside the defined or acceptable upper and lower threshold brightness levels.

It should be appreciated that the duration and extent of electrical stimulation of the plurality of actuating devices or sensors and the resultant direction and type of movement of the actuatable lens of the illuminator(s) is determined based on at least a) the identified region or segment, b) the brightness level (such as the Gaussian local brightness) of the identified region or segment and c) the type of actuatable lens (stiff, flexible or coated) of the concerned illuminator(s) that need to be manipulated. Also, as discussed earlier in the specification with reference to FIGS. 5 through 7 the actuatable lens can be manipulated in one or any combination of X, Y and Z mutually orthogonal directions.

In another embodiment, the actuatable lenses of the illuminators are maneuvered manually by the physician using a plurality of buttons or switches available on the handle of the endoscope (such as the buttons 205 on the handle 204 of the endoscope 202 of FIG. 2).

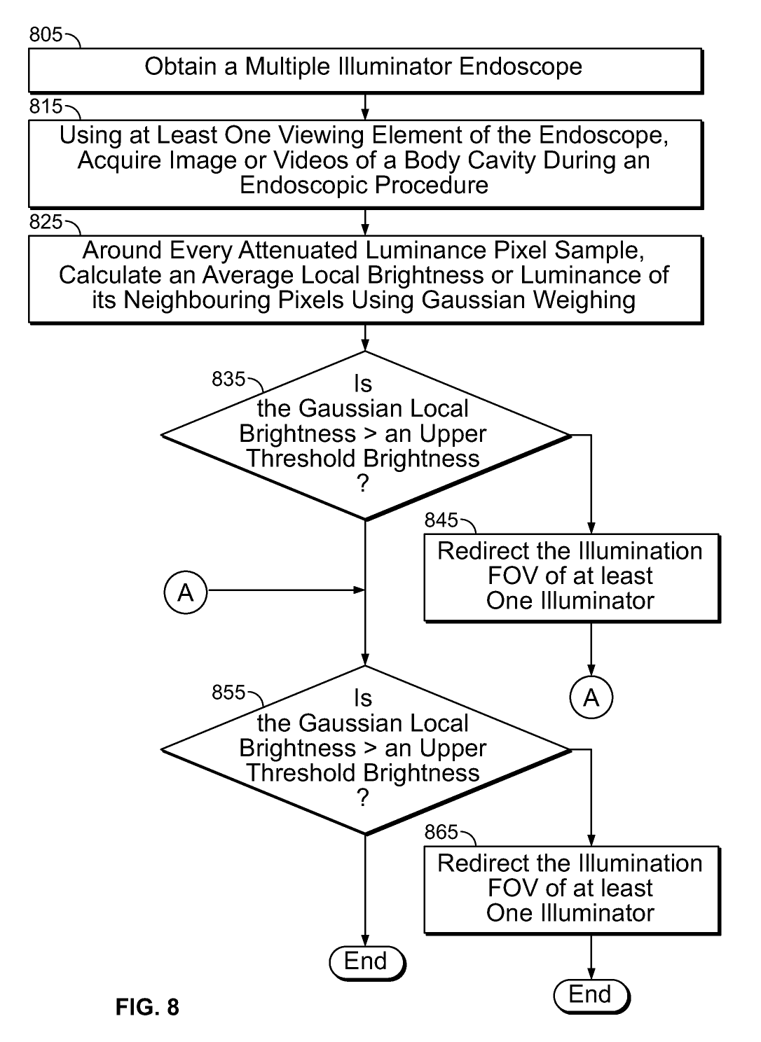

FIG. 8 is a flow chart illustrating a plurality of exemplary steps of a method of controlling distribution of illumination or brightness for a multiple illuminator endoscopic tip of the present specification, in accordance with an embodiment. At step 805 an endoscope of the present specification (such as the endoscopic tip 100 of FIG. 1) is obtained that has a tip section comprising at least one viewing element, a plurality of illuminators associated with the at least one viewing element and configured to illuminate a FOV of the at least one viewing element, wherein at least one of the plurality of illuminators includes an actuatable lens maneuverable using a plurality of transducers (such as piezoelectric transducers or sensors, electrodes, etc.) connected thereto. The endoscope is controlled by a central main control unit. At step 815, the at least one viewing element acquires images or videos of a body cavity during an endoscopic procedure while the plurality of associated illuminators lighten up the FOV of the at least one viewing element. At step 825, the main control unit processes the image or video frame data acquired by the at least one viewing element such that, in one embodiment, around every luminance pixel sample arriving from the at least one viewing element, a Gaussian (averaging process using Gaussian weighing) of its neighborhood pixels is calculated. Hence, the Gaussian is the local brightness around the luminance pixel sample.

At step 835, the Gaussian local brightness values or levels of the acquired image or video frame are compared with an upper threshold brightness level and a lower threshold brightness level. This comparison is used to identify or segment those areas or regions of the acquired image or video frame having Gaussian local brightness levels above the upper threshold brightness level and/or below the lower threshold brightness level. Thus, at step 835, if the Gaussian local brightness level of a region or area is determined to be above the upper threshold brightness level then the region is identified or segmented as being too bright, saturated or over exposed. As a result of this identification, at step 845, the main control unit causes at least one of the plurality of transducers to maneuver the at least one actuatable lens so as to redirect the illumination FOV of the at least one illuminator with reference to the other illuminators till the Gaussian local brightness level of the identified over exposed region falls within the range defined by the upper and lower threshold brightness levels.

At step 855, if the Gaussian local brightness level of a region or area is determined to be below the lower threshold brightness level then the region is identified or segmented as being too dim or under exposed. As a result of this identification, at step 865, the main control unit causes at least one of the plurality of transducers to maneuver the at least one actuatable lens so as to redirect the illumination FOV of the at least one illuminator with reference to the other illuminators till the Gaussian local brightness level of the identified under exposed region falls within the range defined by the upper and lower threshold brightness levels.

The above examples are merely illustrative of the many applications of the system of present specification. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

We claim:

1. A lens actuation system for an endoscope, the system comprising:
at least one viewing element configured to capture images;
a plurality of illuminators configured to illuminate a plurality of fields of view (FOVs) associated with said at least one viewing element;
a controller; and
at least one actuatable lens positioned in front of one of said plurality of illuminators, wherein said at least one actuatable lens is configured to dynamically change an illumination direction of said one of the plurality of illuminators based upon a signal from said controller.

2. The system according to claim 1, wherein said plurality of FOVs partially overlap.

3. The system according to claim 1, comprising an actuatable lens positioned in front of each of said plurality of illuminators.

4. The system according to claim 1, wherein said at least one actuatable lens is a stiff lens positioned proximate to a plurality of electro actuators configured to move said stiff lens.

5. The system according to claim 1, wherein said at least one actuatable lens is a flexible lens positioned proximate to a plurality of actuators configured to deform said flexible lens.

6. The system according to claim 1, wherein said at least one actuatable lens is coated with an electro responsive material that changes its local light refraction index in response to an applied electric field.

7. The system according to claim 1, wherein said at least one actuatable lens dynamically changes an illumination direction by rotating or translating or a combination thereof.

8. The system according to claim 5, wherein said flexible lens is a silicon lens.

9. The system according to claim 5, wherein the deformation of said flexible lens is selected from a group consisting of: contracting, expanding, pulling, pushing and combinations thereof.

10. The system according to claim 6, wherein said electro responsive coating material is selected from a group consisting of: a liquid crystal, an electro responsive polymer, inorganic crystals, metamaterials or combinations thereof.

11. The system according to claim 6, wherein said electric field is applied by a plurality of electrodes.

12. The system according to claim 1, wherein, where light intensity exceeds a predetermined threshold at a certain FOV, due to at least a partial overlap of illumination from more than one of said plurality of illuminators, the at least one actuatable lens is configured to dynamically redirect illumination from one of said plurality of illuminators in order to reduce light intensity at said certain FOV.

13. The system according to claim 1, wherein, where light intensity is below a predetermined threshold at a certain FOV, the at least one actuatable lens is configured to dynamically redirect illumination from at least one of said plurality of illuminators in order to increase light intensity at said certain FOV.

14. The system according to claim 1, wherein said at least one actuatable lens is actuated based on a detection of bright and/or dark areas in said plurality of FOVs.

15. The system according to claim 1, further comprising a user interface configured to allow a user to manually actuate the at least one actuatable lens according to desired light intensity of images presented on a display.

16. The system according to claim 1, further comprising a user interface configured to allow a user to actuate the at least one actuatable lens in order modify a blooming effect, saturation effect, underexposure effect, or overexposure effect.

17. The system according to claim 1, comprising an actuatable lens positioned in front of more than one of said plurality of illuminators.

18. The system according to claim 1, comprising a front viewing element with at least two front illuminators, and a side viewing element with at least two side illuminators, wherein an actuatable lens is positioned in front of each of said two front illuminators and said two side illuminators, to dynamically change an illumination direction of one or both illuminators of said front and side illuminators based upon a signal from said controller.

19. The system according to claim 1, comprising a front viewing element with at least two front illuminators and a side viewing element with at least two side illuminators, wherein an actuatable lens is positioned in front of said front and side viewing elements to dynamically change direction of incoming light beams to said front and side viewing elements based upon a signal from said controller.

20. A method of controlling illumination of an endoscope tip, the method comprising:
providing, at said endocope tip, at least one viewing element configured to capture images, a plurality of illuminators configured to illuminate a plurality of field of views (FOVs) associated with said at least one viewing element and at least one actuatable lens positioned in front at least one of the plurality of illuminators;
receiving, from said at least one viewing element, images of a plurality of FOVs illuminated by said plurality of illuminators; and,
dynamically redirecting illumination by actuating said at least one actuatable lens in order to reduce light intensity of a too bright captured image or increase light intensity of a too dark captured image.
